(19) United States
(12) Patent Application Publication
(10) Pub. No.: US 2012/0095427 A1
(43) Pub. Date: Apr. 19, 2012

(54) EVACUATION DIAPER

(76) Inventor: Luiz DASILVA, Irvington, NJ (US)

(21) Appl. No.: 12/904,810

(22) Filed: Oct. 14, 2010

(51) Int. Cl. A61F 13/15

(52) U.S. Cl. 604/385.13; 604/385.01

(57) ABSTRACT

An evacuation diaper comprising a front panel to cover a pelvis of a wearer, a back panel to fit against a lower back of the wearer, a set of sides between the front panel and the back panel to fit between a pair of legs of a wearer, and an expelling hole located between the front panel and the back panel on a central portion of the evacuation diaper wherein the expelling hole enables the wearer to defecate into a toilet without removing the evacuation diaper. The evacuation diaper encourages children and adults who may suffer from trauma, a developmental or neurological disorder, or another mental condition that hinders them from feeling comfortable with using a restroom, to relieve themselves independently and without the assistance of another person.

### BACKGROUND OF THE INVENTION

1. Field of the Invention

The present invention relates to a diaper to teach children or individuals with learning disabilities to use a toilet without having to remove the diaper.

2. Description of Related Art

As every parent knows potty training is a tiresome and cumbersome task. Accidents, rewards and panic are all terms associated with the parents teaching their children how to live without a diaper. Some children learn right away transitioning into life without diapers quite easily, while others need a bit more coaxing and encouragement. Even though most children learn to use a toilet within the first 4 years of their life, others may need more time or a different approach to normal potty training.

Many children and even adults afflicted with a developmental disorders, autism or even down syndrome may require either more time to learn how to use a toilet or a different approach from the conventional methods of potty training Some of these individuals learn from habit and ritual and do not adapt well to sudden changes or modifications. When trying to teach the person to live without a diaper, they may not associate removal of the diaper with relieving themselves in a toilet. Also they may be frightened when the diaper, a piece of clothing they've worn all their lives, is removed and they are forced to defecate in an unknown and unfamiliar location. In some ways, the diaper may be associated as a type of security blanket wherein the person is comfortable using and wearing it, and to remove it requires serious behavioral and emotional modification.

It would be beneficial in the art to have a diaper which allows a person with developmental or neurological disorders to learn to use a toilet. Also, it would be desirable to have a diaper which allows the wearer to feel like they are wearing a diaper but they are actually relieving themselves into a toilet.

### SUMMARY OF THE INVENTION

The present invention relates to an evacuation diaper to allow individuals with developmental or neurological disabilities to use a toilet. A person wearing the evacuation diaper may be able to sit on the toilet and relieve himself or herself while wearing the diaper. The evacuation diaper comprises a front panel to fit over a pelvis of a wearer, a back panel to cover up to a lower back of the wearer, a set of sides between the front panel and the back panel to fit between a pair of legs of the wearer, and an expelling hole located between the front panel and the back panel on a central portion of the evacuation diaper wherein the expelling hole enables the wearer to defecate into a toilet without removing the evacuation diaper.

Another object of the present invention is to alleviate the stress associate with potty training and using a toilet.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 is a perspective view of an evacuation diaper in accordance with an exemplary embodiment of the present invention; and

FIG. 2 is a perspective view of the backside of the evacuation diaper in accordance with an exemplary embodiment of the present invention.

Like reference numerals refer to like parts throughout the description of several views of the drawings.

### DETAILED DESCRIPTION OF THE DRAWINGS

The present invention relates to a diaper intended to encourage children and adults to use the toilet independently by alleviating the stress associated with transitioning from using a diaper to using a toilet. The wearer may be children or individuals who suffer from trauma, developmental or neurological disorders or mental conditions that hinder them from feeling comfortable when using a restroom. The present invention discloses an evacuation diaper designed with a centralized circular opening at a location where solid and liquid waste may be easily expelled. During initial use, the person may be prompted by their guardian to use the toilet while wearing the evacuation diaper. This may provide the person with a sense of security and normalcy. By not requiring the wearer to remove the evacuation diaper the person may relieve themselves feeling comfortable and confident. After the evacuation diaper is employed, the guardian should continue to encourage and reinforce the behavior repeatedly until the wearer is confident enough to use the lavatory without a diaper at all and without the assistance of another person.

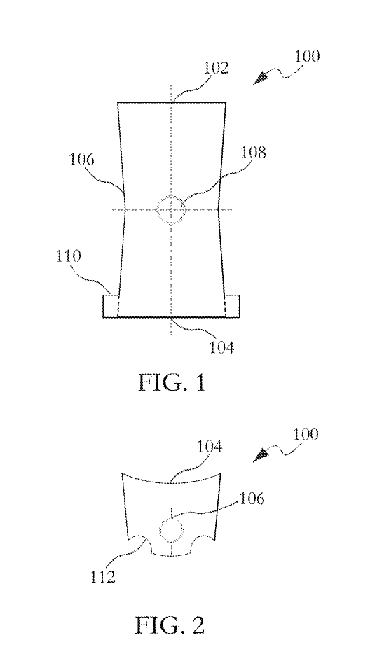

Turning now descriptively to the drawings, referring to FIG. 1, a perspective view of an evacuation diaper (100) is shown in accordance with an exemplary embodiment of the present invention. The evacuation diaper (100) comprises a front panel (102), a back panel (104) and a set of side panels (106) (hereinafter sides). The front panel (102) may fit over a front torso or pelvis of a wearer and wrap around to the sides of the wearer. The back panel (104) may fit over the posterior area of the torso, extending over the buttocks to the small of the back. The sides (106) fit between the legs of the wearer and transition from the front panel (102) to the back panel (104). The sides (106) may curve inward to allow a more comfortable fit for the wearer while moving around, walking and sitting. In a central portion of the evacuation diaper (100) may be an expelling hole (108). The expelling hole (108) may serve as a dumping location or evacuation space through which the wearer may defecate into a toilet. The expelling hole (108) may have a circular or oval shape, and is designed to expose the rectum of the wearer. Along the sides of the back panel (104) may be a set of tabs (110) (hereinafter tabs). The tabs (110) may secure the back panel (104) to the front panel (102) around the torso of the wearer. The tabs (110) may include a sticky material to adhere the tabs (110) onto the front panel (102) or a Velcro piece for attachment.

Referring to FIG. 2, a backside view of the evacuation diaper (100) is shown in accordance with an exemplary embodiment of the present invention. The expelling hole (106) is shown in a central location against the back panel (104). Along the lower portion of the evacuation diaper (100) may be a set of leg holes (112) to accept the legs of the wearer. The leg holes (112) may essentially be the result of the sides wrapping around the upper thigh of the wearer. The legs holes (112) may include an elastic strip sewn into the sides to ensure that the evacuation diaper fits properly between the legs of the wearer.

The evacuation diaper may be disposable or reusable. If the evacuation diaper is reusable it may be composed of a plastic cover with a cloth liner to lie comfortably against the skin of the wearer. If the evacuation diaper is disposable, it may also include the plastic cover but instead may include a disposable fibrous lining with an absorbent under inner layer. Along the upper portions of both the front panel and the back may be an elastic strip to create a secure waistband around the wearer. The evacuation diaper may be available in sizes ranging from toddler to adult to accommodate the various users. Finally, the evacuation diaper may be produced with various designs to appeal to wearers of all ages and developmental stages.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. An evacuation diaper comprising:
a front panel to cover a pelvis of a wearer;
a back panel to fit against a lower back of the wearer;
a set of sides between the front panel and the back panel to fit between a pair of legs of a wearer; and
an expelling hole located between the front panel and the back panel on a central portion of the evacuation diaper wherein the expelling hole enables the wearer to defecate into a toilet without removing the evacuation diaper.

2. The evacuation diaper of claim 1, wherein the back panel includes a set of tabs to secure the back panel to the front panel around a torso of the wearer.

3. The evacuation diaper of claim 2, wherein the set of tabs includes a layer of sticky material to adhere to the front panel.

4. The evacuation diaper of claim 2, wherein the set of tabs comprises a Velcro piece.

5. The evacuation diaper of claim 1, wherein the evacuation diaper is disposable.

6. The evacuation diaper of claim 1, wherein the evacuation diaper is reusable.

7. An evacuation diaper comprising the steps:
securing the evacuation diaper around a torso of a wearer by:
folding a front panel over a pelvis of the wearer;
folding a back panel between a pair of legs and against a lower back of the wearer; and
fastening a set of tab on the back panel of the wearer around the torso to the front panel;
placing the wearer on a toilet;
encouraging the wearer to relieve themselves;
offering positive reinforcement when the wearer uses the toilet; and
repeatedly encouraging the wearer to use the toilet until the wearer learns to use the toilet independently from another person.
